(19) United States
(12) Patent Application Publication
(10) Pub. No.: US 2026/0182725 A1
(43) Pub. Date: Jul. 2, 2026

(54) MULTI- FUNCTIONAL CAP FOR SKINCARE PRODUCT

(71) Applicant: SHENZHEN NUON MEDICAL EQUIPMENT CO., LTD., Shenzhen (CN)

(72) Inventors: Alain Dijkstra, Amstelveen (NL); Li Xiang, Shenzhen (CN); Tang Yuanzhou, Shenzhen (CN); Huang Jianlong, Shenzhen (CN)

(73) Assignee: Shenzhen Nuon Medical Equipment Co., Ltd., Shenzhen (CN)

(21) Appl. No.: 19/433,742

(22) Filed: Dec. 27, 2025

(30) Foreign Application Priority Data
Dec. 31, 2024 (CN) 2024233200205

(51) Int. Cl. A45D 40/26; A61F 7/00; A61N 5/06

(52) U.S. Cl. CPC A45D 40/265; A45D 40/264; A61F 7/007; A61N 5/0616; A45D 2200/155; A45D 2200/205; A61F 2007/0052; A61F 2007/0071; A61F 2007/0087; A61N 2005/0644

(57) ABSTRACT

The present invention discloses a functional cap and storage container assembly designed to enhance product extraction, hygiene, and therapeutic performance. The functional cap includes a detachable upper cap assembly and lower cap assembly, with a cream applicator elastically coupled to the upper cap assembly for controlled axial movement. The lower cap assembly houses stimulation elements such as thermal, phototherapy, microcurrent, magnetotherapy, or ultrasonic devices, enabling sequential skincare application and skin treatment. Advanced sealing structures, including sealing rings and retractable slits, prevent leakage while allowing smooth applicator movement. The upper cap assembly can independently provide phototherapy, with integrated LEDs, circuitry, and a rechargeable battery, leveraging conductive elements in the lower cap for power transfer. Additional embodiments include elastic locking members for controlled access to the skincare product. The system provides a versatile, compact, and user-friendly solution suitable for cosmetic, dermatological, and home-use applications.

### TECHNICAL FIELD

The present invention relates to skincare product packaging and cosmetic storage container technology, and more particularly to a functional cap for skincare products that integrates a cream applicator with an elastic reset mechanism and one or more simulation elements such as heat therapy, phototherapy, or microcurrent therapy.

### BACKGROUND ART

Cream-based and gel-based skincare products are commonly packaged in wide-mouth jars or bottles, where users typically remove the product using their fingers or a simple spatula. Traditional cosmetic containers rely on fixed-length spatulas or manual scooping tools that are either stored separately or attached to the lid. While these basic tools provide convenience, they are limited by their fixed geometry: the spatula is typically shorter than the height of the bottle to avoid interference when the container is closed, resulting in difficulty reaching the bottom and inner sidewalls of the container as the product level decreases. This leads to product residue, waste of formulations, and potential hygiene concerns when users repeatedly reach into the container with their fingers.

Some existing designs attempt to reduce contamination by integrating a spatula into the container lid. However, because these spatulas cannot extend beyond a predetermined length, they still fail to access the deep corners and lower regions of the bottle. When scooping cream from around the jar at an angle, the spatula may not reach the bottom, thus failing to address the problem of cream residue inside the jar. Additionally, separate spatulas or scooping tools may become lost or misplaced when not attached to the container.

Cosmetic applicators have been developed with various configurations to improve the application of cosmetics. Some applicators include spiral-shaped application members that extend from rod-like support members to facilitate the scooping and spreading of cosmetics. Other designs incorporate cosmetic brushes with stems that can move within supporting tubes, where springs bias the stems in particular directions. These designs address issues such as bristle tangling in high-viscosity liquids and provide mechanisms for the applicator to retract when the cap is engaged with the container.

Skincare product covers have also been developed that incorporate storage positions for defrosting spoons, magnetic attraction elements for securing the spoons, and wireless charging capabilities for batteries within the spoons. Some cream bottle designs include introducing mechanisms that can be rotated to expose or conceal an introducing surface, where the introducing mechanism may include phototherapy lamps, heating elements, and massage balls. Container covers have been developed with mounting seats that can move within accommodating cavities, with elastic members pushing the mounting seats toward particular positions, and with locking positions and guide structures to control the movement of the mounting seats.

Despite these developments, existing skincare caps do not adequately integrate therapeutic components, such as heating, cooling, microcurrent, or phototherapy elements, in a manner that allows for convenient sequential application of skincare products and skin stimulation. Consumers are often required to use separate devices for skin stimulation, which is inconvenient and time-consuming, and increases the likelihood of losing or misplacing small accessories. There remains a desire in the market for a multifunctional cap system that enables full extraction of skincare products while also delivering therapeutic benefits in a compact, hygienic, and user-friendly manner.

The present invention addresses these limitations by providing a functional cap equipped with an elastic reset mechanism that enables the cream applicator to automatically extend when the cap is opened, allowing the applicator to reach the bottom and sidewalls of the bottle and significantly reducing product residue. The detachable lower cap assembly incorporates optional stimulation elements such as heat therapy, cold therapy, phototherapy, or microcurrent treatment, allowing immediate skin stimulation after product application. The design integrates sealing structures, quick-release mechanisms, and guided sliding components to enhance usability, prevent leakage, maintain cleanliness, and improve skincare absorption. Thus, the invention overcomes the deficiencies of existing containers and offers a compact, multifunctional, and user-friendly solution tailored to modern skincare demands.

### OBJECTS OF THE INVENTION

Some of the objects of the invention are as follows:
An object of the present invention is to provide a functional cap with a storage container comprising a detachable upper and lower cap assembly, wherein a cream applicator is elastically coupled to the upper cap assembly for controlled axial movement, enabling efficient extraction and application of skincare products from the container.

Another object of the present invention is to provide a cream applicator wherein the lower cap assembly integrates stimulation elements such as thermal, phototherapy, microcurrent, magnetotherapy, or ultrasonic devices, allowing sequential skincare application and therapeutic treatment in a single operation.

Another object of the present invention is to provide a functional cap system with advanced sealing structures, including retractable slits, dynamic sealing rings, or elastic locking members, to prevent leakage of the skincare product while permitting smooth movement of the cream applicator.

Another object of the present invention is to enable the upper cap assembly to operate independently as a phototherapy device, incorporating LEDs, circuitry, and rechargeable batteries, while utilizing conductive elements in the lower cap assembly for charging and microcurrent delivery.

Another object of the present invention is to integrate an elastic locking member operably coupled to a pressing member, allowing controlled access to the skincare product only when manual pressure is applied, thereby improving hygiene, safety, and user control.

Another object of the present invention is to provide a modular construction wherein the upper cap assembly and its internal components, such as the sleeve, slider, and elastic reset member, can be individually manufactured, serviced, or replaced, enhancing durability and customization.

Another object of the present invention is to provide a skincare applicator system suitable for various industrial and consumer applications, including home-use, professional dermatology, cosmetic packaging, portable beauty kits, and multifunctional skincare devices.

Another object of the present invention is to enhance product utilization, user convenience, and therapeutic efficiency through the combination of guided sliding mechanisms, detachable cap assemblies, integrated stimulation elements, and optional pre-treatment light-therapy elements.

Another object of the present invention is to provide a compact, ergonomic, and versatile applicator system capable of accommodating diverse container-neck formats and supporting scalable mass manufacturing using conventional injection-molding and electronic-assembly processes.

### SUMMARY OF THE INVENTION

According to a first aspect of the present invention, a functional cap for a storage container is provided. The functional cap comprises: a lower cap assembly defining a clearance hole extending therethrough and housing at least one stimulation element; an upper cap assembly detachably connected to the lower cap assembly, the upper cap assembly positioned to cover the clearance hole when attached to the lower cap assembly; a leakage protection member to form a fluid-resistant seal between a wall of the clearance hole and the upper cap assembly; and a cream applicator extending through the clearance hole and supported by the upper cap assembly, wherein the cream applicator is positioned to contact a skincare product stored in the storage container when the functional cap is mounted on the storage container, and wherein the lower cap assembly is configured to provide one or more therapy to a user through the at least one stimulation element.

In one embodiment of the invention, the lower cap assembly has opposite first and second ends, the clearance hole extending between the first end and the second end.

In one embodiment of the invention, at least one stimulation element is disposed at the first end of the lower cap assembly, and the upper cap assembly covers the first end.

In one embodiment of the invention, the leakage protection member is a sealing ring disposed at the clearance hole, the sealing ring comprises an annular elastomeric body configured to inhibit leakage of the skincare product.

In one embodiment of the invention, the cream applicator includes an applicator surface selected from a spatula, pad, brush, or scoop.

In one embodiment of the invention, the leakage protection member comprises a retractable structure with one or more slits configured to allow controlled deformation.

In one embodiment of the invention, the leakage protection member comprises an elastic locking member disposed at the clearance hole and configured to selectively open and close the clearance hole; and a pressing member operably coupled to the elastic locking member, wherein actuation of the pressing member causes the elastic locking member to deform and expose the clearance hole, and release of the pressing member causes the elastic locking member to return to a closed state covering the clearance hole.

In one embodiment of the invention, the upper cap assembly includes a rechargeable battery and a pair of charging electrodes disposed on a bottom surface of the upper cap assembly; and the lower cap assembly includes a pair of conductive elements electrically connected to power circuitry housed within the lower cap assembly, wherein the charging electrodes are positioned to electrically interface with the pair of conductive element when the upper cap assembly is coupled to the lower cap assembly, such that the conductive element function as charging terminals to transfer electrical power to the rechargeable battery of the upper cap assembly.

In one embodiment of the invention, the functional cap further comprises an elastic reset member disposed within the upper cap assembly and operably connected to the cream applicator.

In one embodiment of the invention, the elastic reset member comprises a spring or elastomeric element, which permits axial displacement of the cream applicator relative to the upper cap assembly.

In one embodiment of the invention, the upper cap assembly comprises an upper cover body, a sleeve, and a slider, the upper cover body having a slot, one end of the sleeve being inserted into the slot, the elastic reset member and the slider being located within the sleeve, the slider being slidably engaged with the sleeve, and one end of the cream applicator being fixed to the slider.

In one embodiment of the invention, an inner wall of the sleeve is provided with a plurality of guide ribs extending along an axial direction of the sleeve, and a circumferential surface of the slider is provided with a plurality of guide grooves configured to receive the plurality of guide ribs in a one-to-one correspondence.

According to a second aspect of the present invention, a skincare product dispensing and treatment system is provided. The system comprising: a functional cap removably coupled to the storage container, wherein the functional cap includes a lower cap assembly housing at least one stimulation element, an upper cap assembly detachably connected to the lower cap assembly, a leakage protection member disposed at a clearance hole of the lower cap assembly, and a cream applicator supported by the upper cap assembly and extending through the clearance hole into the storage container, wherein the lower cap assembly is separable from the storage container and configured for provide one or more therapy to a user through the at least one stimulation element.

In one embodiment of the invention, the leakage protection member is a sealing ring configured to seal a gap between a wall of the clearance hole and the upper cap assembly.

In one embodiment of the invention, the lower cap assembly further comprises a first cover and a second cover that cooperate to form a mounting cavity, the mounting cavity is configured to accommodate a circuit board and a battery electrically connected to at least one stimulation element.

In one embodiment of the invention, the at least one stimulation element comprises a heating resistor mounted on the circuit board and a conductive element fixed in a through hole of the first cover, the conductive element being positioned directly opposite the heating resistor to transfer heat generated by the heating resistor to a user's skin.

In one embodiment of the invention, the first cover is formed of a light-transmitting material, and at least one stimulation element comprises a light-emitting element mounted on the circuit board and configured to emit therapeutic light through the first cover.

According to a third aspect of the present invention, a method of applying a skincare product and providing skin treatment using a functional cap and a storage container is provided. The method comprising: providing an upper cap assembly in the functional cap, the upper cap assembly comprising a cream applicator to contact a skincare product stored in the storage container; providing a lower cap assembly in the functional cap, the lower cap assembly having a clearance hole for allowing the cream applicator to pass through, the lower cap assembly housing at least one stimulation element; providing a leakage protection member configured to close the clearance hole to prevent leakage of the skin care product; removing the functional cap from the storage container; retrieving the skincare product from the storage container using the cream applicator; applying the skincare product to the skin using the cream applicator; and applying one or more therapies to a user using at least one stimulation element of the lower cap assembly.

In one embodiment of the invention, applying one or more therapies is performed after application of the skincare product.

In one embodiment of the invention, the cream applicator is biased toward an extended position by an elastic reset member.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “several” means more than one, unless otherwise specified.

In the context of the specification, the term “therapy device”, or “physiotherapy device” refers to the device of the present invention configured to perform phototherapy, thermal therapy, microcurrent therapy, magnetotherapy, ultrasonic wave therapy, or combined treatment.

In the context of the specification, the term “stimulation element” refers broadly to any component, module, or structure configured to apply a therapeutic or cosmetic stimulus to a user's skin or tissue. Stimulation elements may include, but are not limited to, phototherapy elements, massage elements, microcurrent electrodes, ultrasonic transducers, heating elements, cooling elements, PEMF coils, or combinations thereof. The PEMF coil refers to any structure configured to generate pulsed or modulated electromagnetic fields for therapeutic purposes. The PEMF coil may be integrated with or share components with the wireless charging assembly, such as using the same coil for both charging and electromagnetic therapy functions.

In the context of the specification, the terms “phototherapy element”, “light-emitting element”, and “light-emitting unit” encompass any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infrared spectra.

In the context of the specification, the term “massage element” refers to any component adapted to apply mechanical stimulation to the skin, including rotating rollers, kneading members, vibrating members, or reciprocating structures. The massage element may be fixed, detachable, or mounted for rotation or vibration relative to the housing.

In the context of the specification, the term “microcurrent element” refers to any electrode or conductive structure configured to deliver a controlled electrical signal to the user's skin. Such elements may include paired electrodes, conductive surfaces, or pads connected to a circuit board for generating microcurrent, galvanic current, or equivalent electrical therapy.

In the context of the specification, the term “housing” is intended to cover any casing, enclosure, or structural body that contains or supports components of the device. The housing may include a handle portion, head portion, or other segments, and may be made from polymeric, metallic, composite, or other suitable materials.

In the context of the specification, the terms “liquid container,” “liquid storage chamber,” or “reservoir” refer to a chamber or container configured to hold skincare liquid.

In the context of the specification, the term “heating element,” “temperature control element,” or “thermal element” refers to any suitable device or structure for heating or cooling the treatment surface or liquid.

In the context of the specification, the term “light-emitting element,” “phototherapy component,” or “light-transmitting surface” refers to a component configured to emit light for skin treatment.

In the context of the specification, the term “sealing pad,” “retaining ring”, or “gasket” refers to a component configured to provide a sealed connection between structural elements, preventing liquid or mist leakage.

In the context of the specification, the term “leakage protection member” refers broadly to any component or structure configured to prevent leakage of skincare product through the clearance hole of the lower cap assembly. The leakage protection member may include, but is not limited to, a sealing ring, a retractable structure with one or more slits, an elastic membrane, a flexible gasket, or any combination thereof. The leakage protection member may be disposed at or within the clearance hole and may be configured to form a fluid-resistant seal between the wall of the clearance hole and the upper cap assembly or the cream applicator.

In the context of the specification, the term “LED module” refers to one or more light-emitting diode (LED) elements that are electrically connected and configured to emit light of specific wavelengths suitable for therapeutic purposes. The LED element may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photo-biomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 is a perspective view of a functional cap and a storage container to contain skincare products, in accordance with an embodiment of the present invention.

FIG. 2 is a top view of the functional cap with the point A-A, in accordance with an embodiment of the present invention.

FIG. 3 is a cross-sectional view of the functional cap and the storage container along point A-A shown in FIG. 2, in accordance with an embodiment of the present invention.

FIG. 4 is an exploded view of the functional cap and the storage container, in accordance with an embodiment of the present invention.

FIG. 5 is an exploded view of an upper cap assembly, in accordance with an embodiment of the present invention.

FIG. 6 is a perspective view of a sleeve, in accordance with an embodiment of the present invention.

FIG. 7 is an exploded view of a lower cap assembly, in accordance with an embodiment of the present invention.

FIG. 8A and FIG. 8B show bottom view of the bottom surface of a sealing ring showing slits/cuts in different configurations, in accordance with an embodiment of the present invention.

FIG. 9 is a top surface of the lower cap assembly integrated with a retractable sealing member, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a multifunctional cap and storage container assembly. The assembly may also be referred to as a skincare product dispensing and treatment system, wherein the functional cap enables both dispensing of skincare product from the storage container and therapeutic treatment of the user's skin.

The present invention relates to a functional cap and storage container assembly, configured to improve product extraction, hygiene, and therapeutic performance. The invention discloses a functional cap comprising a detachable upper cap assembly and a lower cap assembly, wherein a cream applicator is elastically coupled to the upper cap assembly via an elastic reset member. The cream applicator is capable of axial displacement relative to the upper cap assembly, allowing it to reach the bottom and peripheral surfaces of the storage container, thereby minimizing residual product and enhancing overall utilization of the skincare formulation. The lower cap assembly is configured to house one or more stimulation elements, including thermal, phototherapy, microcurrent, magnetotherapy, and ultrasonic wave therapy elements, enabling the user to perform sequential skincare application and stimulation, thereby improving formulation absorption and treatment efficacy. The functional cap further incorporates advanced sealing structures, including sealing rings and retractable slits or cuts, to prevent unintended leakage while permitting smooth movement of the cream applicator.

In various embodiments, the upper cap assembly may be modular or integrally formed, comprising components such as a sleeve, slider, and elastic reset member, which allow individual manufacturing, servicing, and replacement. The lower cap assembly may include circuit boards, batteries, and thermal or light-emitting elements, with mounting cavities and positioning grooves to ensure structural stability. The upper cap assembly can additionally function as an independent phototherapy device with integrated LEDs, circuitry, and rechargeable batteries, leveraging conductive elements in the lower cap assembly for power transfer and microcurrent therapy. Alternate configurations include elastic locking members with pressing mechanisms to selectively open and close the clearance hole, enhancing leakage prevention and user control. Collectively, these embodiments provide a versatile, compact, and user-friendly skincare applicator system suitable for mass manufacturing and wide industrial application across personal care, cosmetic, and therapeutic devices.

The functional cap of the present invention generally comprises an upper cap assembly, a lower cap assembly, a cream applicator, an elastic reset member, and one or more sealing structures. The upper cap assembly and the lower cap assembly are detachably connected to one another, allowing the lower cap assembly to be separated and used independently as a handheld stimulation device. The cream applicator extends through a clearance hole formed in the lower cap assembly and is operably connected to the upper cap assembly via the elastic reset member. This configuration enables the cream applicator to move axially relative to the upper cap assembly by a predetermined stroke, thereby allowing the applicator to reach the bottom and sidewalls of a storage container when the functional cap is removed from the container.

In operation, when the functional cap is mounted on a storage container in a closed state, the distal end of the cream applicator contacts the bottom surface of the container, compressing the elastic reset member and storing elastic potential energy. When the functional cap is opened or removed from the container, the elastic reset member releases its stored energy and urges the cream applicator toward an extended position. This automatic extension mechanism allows the cream applicator to access regions of the container that would otherwise be difficult to reach with a fixed-length applicator, thereby reducing product residue and improving overall utilization of the skincare formulation.

The lower cap assembly is configured to house one or more stimulation elements, which may include thermal elements for heat therapy or cold therapy, phototherapy elements for light-based treatment, microcurrent elements for electrical stimulation, ultrasonic wave therapy elements for mechanical stimulation, magnetotherapy elements for magnetic field therapy, or combinations thereof. By integrating these stimulation elements within the lower cap assembly, the functional cap enables the user to perform sequential skincare application and therapeutic treatment in a single operation. After scooping and applying the skincare product using the cream applicator, the user may detach the lower cap assembly and use the stimulation elements to massage or treat the skin, thereby promoting absorption of the applied product and enhancing therapeutic efficacy.

The functional cap further incorporates a leakage protection member to prevent leakage of the skincare product while permitting smooth movement of the cream applicator. The leakage protection member may take various forms depending on the desired sealing performance and operational characteristics. In one embodiment, the leakage protection member is a sealing ring disposed between the upper cap assembly and the wall of the clearance hole to seal the gap therebetween. In another embodiment, the leakage protection member comprises a retractable structure with one or more slits that elastically deform to permit passage of the cream applicator and return to a closed state when the applicator is withdrawn. In some embodiments, the leakage protection member is an elastic locking member configured to selectively open and close the clearance hole. The leakage protection member forms a fluid-resistant seal that inhibits unintended discharge of skincare product from the storage container.

The upper cap assembly may comprise an upper cover body, a sleeve, and a slider, wherein the sleeve is inserted into a slot formed in the upper cover body, and the slider is slidably engaged within the sleeve. The elastic reset member is disposed within the sleeve and biases the slider toward an extended position. The cream applicator is fixed to the slider such that movement of the slider causes corresponding movement of the cream applicator. Guide ribs on the inner wall of the sleeve and corresponding guide grooves on the slider ensure stable and guided axial movement. A stop flange on the sleeve prevents the slider from sliding out, and limiting protrusions and grooves provide secure engagement between the sleeve and the upper cover body.

The upper cap assembly and the lower cap assembly may be detachably connected via a quick-release structure, such as a card slot and guide slot arrangement that engages with a latch on the lower cap assembly upon rotation. This configuration allows the user to quickly and conveniently separate the assemblies for independent use. In some embodiments, the upper cap assembly may itself function as an independent phototherapy device, incorporating LEDs, circuitry, and a rechargeable battery. Conductive elements in the lower cap assembly, such as conductive elements, may serve dual functions as charging terminals and microcurrent electrodes.

The modular construction of the functional cap allows individual components such as the sleeve, slider, elastic reset member, and stimulation elements to be separately manufactured, serviced, or replaced. This modularity enhances durability, facilitates customization, and supports scalable mass manufacturing using conventional injection-molding and electronic-assembly processes. The functional cap is compatible with various container-neck formats and is suitable for a wide range of applications, including home-use skincare, professional dermatology, cosmetic packaging, portable beauty kits, and multifunctional skincare devices.

The following detailed description, with reference to the accompanying drawings, provides specific structural and operational details of various embodiments of the functional cap and storage container assembly. It should be understood that the embodiments described herein are exemplary and that modifications, variations, and equivalent arrangements may be made without departing from the scope of the invention as defined by the appended claims.

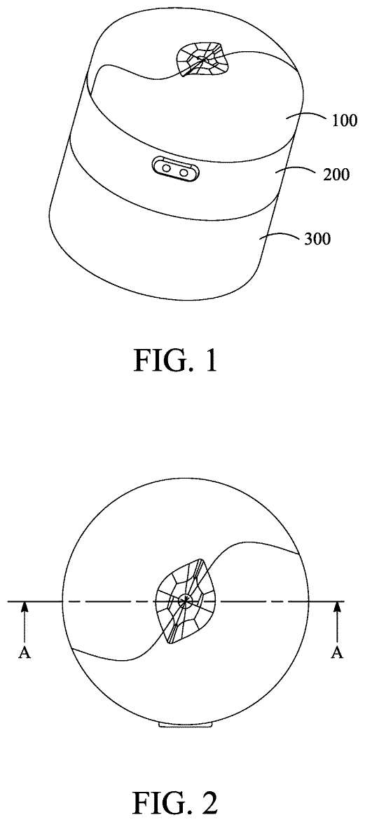

Referring to FIGS. 1 to 4, in an embodiment of the present invention, a functional cap with a storage container 300 is provided. The functional cap comprises an upper cap assembly 100, a lower cap assembly 200, a cream applicator 138 which can be a scoop, a brush, a spatula, an applicator wand, a paddle, a scoop blade, a cosmetic spoon, a dropper, a pipette, a swab, or any equivalent applicating implement capable of transferring skincare products from the storage container 300 to the skin, and an elastic reset member 132. The lower cap assembly 200 is detachably coupled to the upper cap assembly 100. A clearance hole 206 is formed in the lower cap assembly 200, within which one or more stimulation elements can be arranged, such as a thermal element, a light-emitting element, a microcurrent element, a magnetotherapy element, an ultrasonic wave therapy element, etc. The cream applicator 138 extends through the clearance hole 206. The elastic reset member 132, which can be a spring, operably connects the upper cap assembly 100 to the cream applicator 138, enabling the cream applicator 138 to move by a predetermined stroke relative to the upper cap assembly 100 along the axial direction of the clearance hole 206.

In operation, the cream applicator 138 is capable of axial displacement within a preset range as it is elastically coupled to the upper cap assembly 100 via the elastic reset member 132. When the functional cap is mounted onto a storage container 300 and is in the closed state, the distal end of the cream applicator 138 abuts the bottom surface of the storage container 300. This contact compresses the elastic reset member 132, which consequently stores elastic potential energy and exerts a downward biasing force on the cream applicator 138. When the functional cap is opened, the cream applicator 138 is no longer constrained by the bottle bottom. The elastic reset member 132 then returns to its undeformed state, driving the cream applicator 138 downward and thereby increasing its exposed length.

With the elongation of the cream applicator 138, the user can conveniently scoop out the skincare product from the storage container 300. The extended length allows the cream applicator 138 to reach the bottom and peripheral inner surfaces of the storage container 300. This configuration effectively minimizes residual product and enhances overall utilization of the skincare formulation.

Furthermore, the lower cap assembly 200 is configured to be detachable from the upper cap assembly 100 and may be used independently as a physiotherapy device, thereby improving hand-held operability. By integrating the stimulation element within the functional cap and enabling its use in combination with the cream applicator 138, the device allows the user to perform skin stimulation immediately after applying a skincare product with the cream applicator 138. This sequential application enhances the absorption of cream-based skincare formulations. Additionally, locating the stimulation element within the functional cap prevents accidental loss of the stimulation component.

When the lower cap assembly 200 is detached from the upper cap assembly 100, the portion of the cream applicator 138 that would otherwise be obstructed is reduced, since the cream applicator 138 no longer extends through the clearance hole 206 of the lower cap assembly 200. The removal of the lower cap assembly 200 also eliminates interference between the lower cap assembly 200 and the upper cap assembly 100. As a result, during use, the upper cap assembly 100 can be brought into closer proximity to the storage container 300, allowing the cream applicator 138 to access the bottom and inner sidewalls of the storage container 300. This configuration further reduces the likelihood of residual skincare products remaining within the storage container 300.

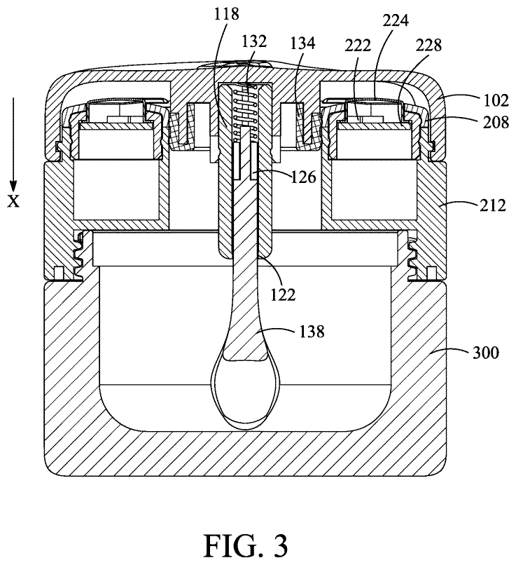

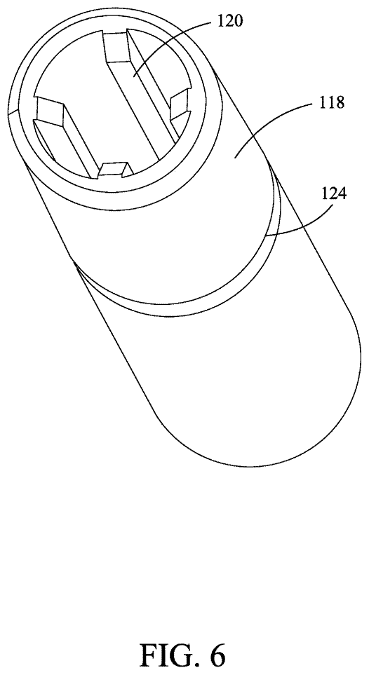

Referring to FIGS. 3, 5, and 6, in an embodiment, the X direction corresponds to the vertical direction extending from top to bottom. In addition, the functional cap further comprises a leakage protection member, and the upper cap assembly 100 is configured to cap the clearance hole 206. In this embodiment, the leakage protection member is a sealing ring 134 that seals the gap between the wall of the clearance hole 206 and the upper cap assembly 100. The sealing ring 134 comprises an annular elastomeric body configured to inhibit leakage of the skincare product. Accordingly, when the functional cap is separated into the upper cap assembly 100 and the lower cap assembly 200, leakage through the clearance hole 206 is prevented, thereby effectively inhibiting unintended discharge of skincare product from the storage container 300.

In an embodiment, the upper cap assembly 100 comprises an upper cover body 102, a sleeve 118, and a slider 126. The upper cover body 102 is formed with a slot 104, into which one end of the sleeve 118 is inserted. The elastic reset member 132 and the slider 126 are disposed within the interior of the sleeve 118, and the slider 126 is slidably coupled to the sleeve 118. One end of the cream applicator 138 is fixed to the slider 126. The elastic reset member 132 is positioned at the end of the slider 126 opposite the cream applicator 138.

When the sleeve 118 is configured as a through-end structure, one end of the elastic reset member 132 abuts the upper cover body 102, while the opposite end abuts the slider 126. When the sleeve 118 is configured as a closed-end structure, one end of the elastic reset member 132 abuts the closed end of the sleeve 118, and the opposite end similarly abuts the slider 126.

The slider 126 is further provided with a mounting hole 128. The end of the cream applicator 138 extends through the mounting hole 128 and is exposed outside the slider 126. The elastic reset member 132 can be a coil spring disposed around the end portion of the cream applicator 138 to maintain proper positioning of the spring. The elastic reset member 132 may alternatively be implemented as a metal spring, a torsion spring, or other elastic biasing component.

In operation, the slider 126 is configured to move axially within the sleeve 118. When the slider 126 is displaced upward, the elastic reset member 132 is compressed. When the lower end of the cream applicator 138 is no longer subjected to an upward resisting force, the elastic reset member 132 recovers its elastic deformation and urges the slider 126 downward, thereby driving the cream applicator 138 downward. The cream applicator 138 can be a scoop, a brush, a spatula, an applicator wand, a paddle, a scoop blade, a cosmetic spoon, a dropper, a pipette, a swab, etc. The cream applicator 138 includes an applicator surface at its distal end, which may be configured as a spatula surface, a pad surface, a brush surface, a scoop surface, or other suitable surface for retrieving and applying skincare products.

The upper cap assembly 100 can be disassembled into the upper cover body 102, the sleeve 118, and the slider 126, and each component can be individually manufactured, serviced, or replaced. This modular arrangement allows each structure to be formed from different materials according to functional requirements. For example, the upper cover body 102 may be fabricated from a lightweight plastic material to reduce overall weight, while the sleeve 118 and the slider 126 may be formed from abrasion-resistant materials to minimize wear resulting from repeated sliding engagement.

In an alternative embodiment, the upper cover body 102 and the sleeve 118 may be integrally formed as a single piece, such as by injection-molding a unitary plastic component, thereby eliminating subsequent assembly steps.

Furthermore, to prevent the slider 126 from tilting or becoming misaligned relative to the sleeve 118 during axial movement, the inner wall of the sleeve 118 is provided with a plurality of guide ribs 120. The guide ribs 120 are circumferentially spaced from one another and extend in the axial direction of the sleeve 118. Correspondingly, the circumferential surface of the slider 126 is formed with a plurality of guide grooves 130, each configured to slidably receive one of the guide ribs 120, thereby ensuring stable and guided movement of the slider 126.

The sleeve 118 is further provided with a stop flange 122 at an end opposite the slot 104. The stop flange 122 is arranged on the inner wall of the sleeve 118 and surrounds the cream applicator 138. The stop flange 122 prevents the slider 126 from sliding out of the sleeve 118 and additionally functions to reduce or prevent skincare product from entering the interior of the sleeve 118. To facilitate the replacement of the sleeve 118, the sleeve 118 is detachably connected to the upper cover body 102. The detachable connection may be achieved through various fastening mechanisms, including but not limited to snap-fit engagement or threaded coupling.

In an embodiment, the slot 104 is formed with a limiting protrusion 116 on its inner wall, while the outer wall of the sleeve 118 is provided with a corresponding limiting groove 124 configured to engage with the limiting protrusion 116. In certain embodiments, the limiting protrusion 116 and the limiting groove 124 may each be formed as annular structures to provide a secure and uniform snap-fit connection.

Furthermore, the upper cover body 102 is formed with a mating flange 106 on the side facing the lower cap assembly 200. The mating flange 106 is annular, and the inner space it surrounds defines the slot 104. The distal end of the mating flange 106, i.e., the end opposite the main portion of the upper cover body 102, is provided with a notch 108. The mating flange 106 functions as a reinforcing rib to enhance the structural rigidity of the upper cover body 102. In addition, the presence of the notch 108 enables the mating flange 106 to locally deform elastically or plastically when the sleeve 118 is inserted into the slot 104, thereby facilitating the insertion process. Once insertion is complete, the mating flange 106 returns to its original shape or transitions to a stable deformed state at the notch 108, thereby tightly clamping and securing the sleeve 118.

Optionally, two notches 108 may be formed on the mating flange 106 in positions arranged symmetrically about the central axis. In other embodiments, the number of notches 108 is not limited and may include one, two, or more according to design requirements.

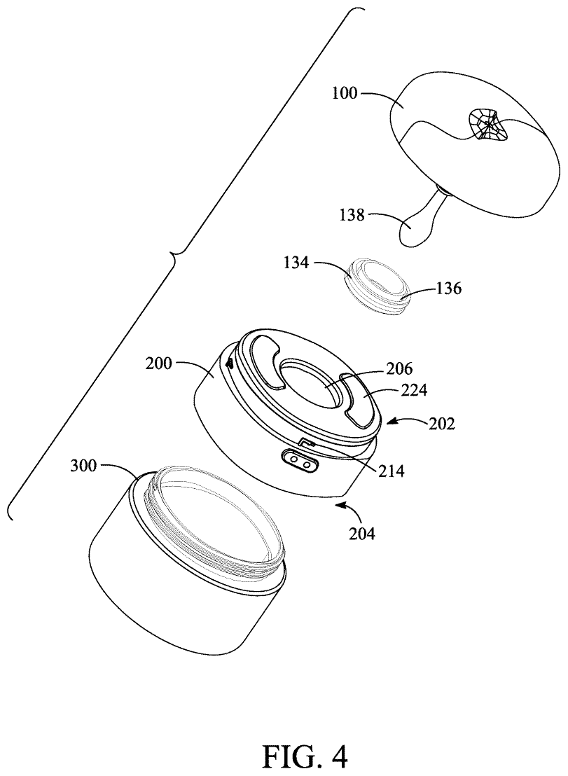

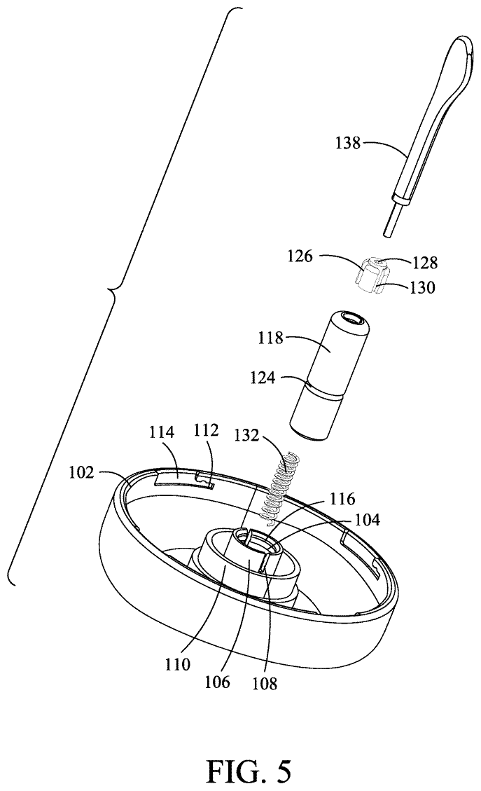

Referring to FIGS. 4 and 5, the upper cover body 102 is further provided with a fixing flange 110 on the side facing the lower cap assembly 200. The fixing flange 110 is annular and is arranged at a position radially outward of the mating flange 106 with a spacing therebetween. The sealing ring 134 is formed with an annular fixing groove 136 configured to receive the fixing flange 110 in an inserted manner. When assembled, the sealing ring 134 and the fixing flange 110 extend into the clearance hole 206, and the outer circumferential surface of the sealing ring 134 sealingly abuts against the wall defining the clearance hole 206.

To further enhance sealing performance, the outer circumferential surface of the sealing ring 134 is provided with a plurality of sealing protrusions. These protrusions are axially spaced apart along the direction of the clearance hole 206 and are configured to contact the inner wall of the clearance hole 206. Owing to their small size, the sealing protrusions readily deform, and the gaps between adjacent protrusions provide compression space during deformation. This structure enables the protrusions to conform more effectively to the hole wall, thereby improving sealing engagement. Moreover, the presence of multiple protrusions forms a multi-stage sealing structure, resulting in substantially improved overall sealing performance.

In other embodiments, the sleeve 118 and the slider 126 may be omitted entirely. In such configurations, the end of the cream applicator 138 is directly inserted into the slot 104 of the upper cover body 102 and is slidably engaged with the slot 104, while the elastic reset member 132 is also accommodated within the slot 104 to provide the necessary elastic biasing function.

In an embodiment, the lower cap assembly 200 includes a first end 202 and a second end 204 positioned opposite each other, with the clearance hole 206 extending through the first end 202 and the second end 204. A stimulation element is disposed at the first end 202, and the upper cap assembly 100 is configured to cover the first end 202 to conceal and protect the stimulation element. As a result, the stimulation element is shielded from external impact or mechanical damage without requiring an additional protective cap. Moreover, the upper cap assembly 100 and the lower cap assembly 200 are arranged vertically, with the lower cap assembly 200 positioned between the upper cap assembly 100 and the storage container 300. When the lower cap assembly 200 is removed, the upper cap assembly 100 can move closer to the storage container 300, thereby enabling the cream applicator 138 to access and contact the bottom surface of the storage container 300 more effectively.

The upper cap assembly 100 and the lower cap assembly 200 are detachably connected. In an embodiment, a quick-release structure is adopted to facilitate the convenient removal of the lower cap assembly 200.

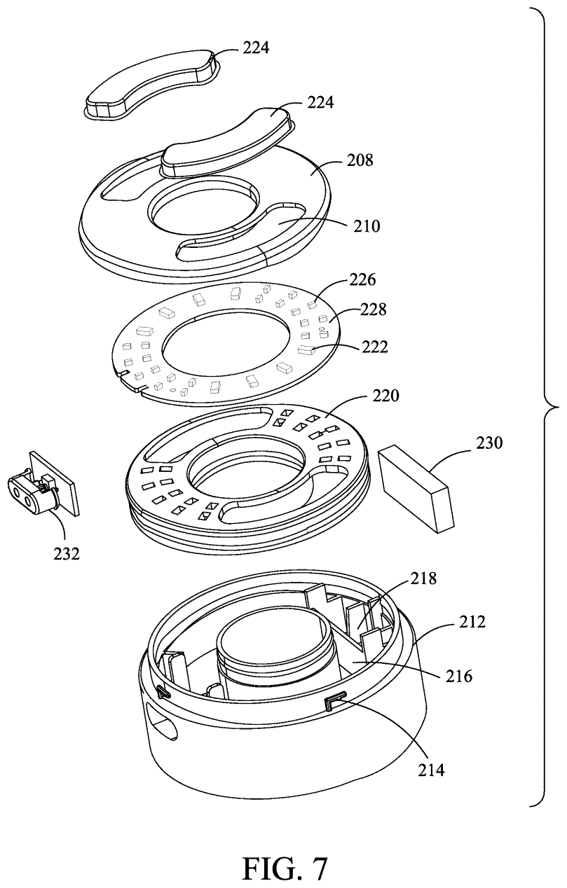

Referring to FIGS. 5 and 7, in an embodiment, the upper cap assembly 100 is provided with an interconnected card slot 112 and a guide slot 114, while the lower cap assembly 200 includes a latch 214. The end of the upper cap assembly 100 is configured to sleeve over the corresponding end of the lower cap assembly 200. Upon rotation of the upper cap assembly 100 relative to the lower cap assembly 200, the latch 214 moves from the guide slot 114 into engagement with the card slot 112.

Further, the card slot 112 and the guide slot 114 are formed on the inner peripheral wall of the upper cap assembly 100, with the guide slot 114 extending downward and opening through the lower edge of the upper cap assembly 100. The latch 214 is formed on the outer peripheral wall of the lower cap assembly 200. During assembly, the latch 214 is first inserted upward into the guide slot 114. Subsequent rotation of the upper cap assembly 100 causes the latch 214 to slide generally horizontally along the guide slot 114 until it enters and locks within the card slot 112. The guide slot 114 thus provides a guiding pathway for engagement of the latch 214 with the card slot 112. The card slot 112 is preferably narrow to facilitate secure retention of the latch 214.

It should be understood that the structural forms of the latch 214 and the card slot 112 are not limited to the above arrangement. For example, in some embodiments, the latch may directly engage the card slot without requiring rotational movement of the upper cap assembly 100.

The upper cap assembly 100 can be provided with multiple card slots 112 and multiple guide slots 114, and the lower cap assembly 200 can integrate multiple corresponding latches 214 to achieve a more stable and evenly distributed locking structure.

In an embodiment, the upper cap assembly 100 and the lower cap assembly 200 may alternatively be connected by a direct inner-outer sleeve engagement structure. In an embodiment, the upper cap assembly 100 and the lower cap assembly 200 can be detachably fixed through magnetic attraction.

The stimulation element may include any one, or any combination, of a thermal element, a phototherapy element, a magnetotherapy element, an ultrasonic wave therapy element, a microcurrent therapy element, etc.

In an embodiment, the stimulation element can be a thermal element that includes a heating resistor 222 and a conductive element 224. When the heating resistor 222 is energized, thermal energy is generated and rapidly transferred to the conductive element 224, which subsequently delivers heat to the user's skin. In an embodiment, the thermal element can employ a semiconductor heating/cooling component in place of the heating resistor 222. The semiconductor heating/cooling component may be a thermoelectric cooler, such as a Peltier device, which operates based on the Peltier effect to transfer heat from one side of the device to the other when an electric current is applied. The thermoelectric cooler includes a cold side and a hot side opposite the cold side. In a cooling configuration, the cold side of the thermoelectric cooler may be positioned adjacent to or in thermal contact with the conductive element 224, such that when the thermoelectric cooler is energized, heat is absorbed from the conductive element 224 and transferred to the hot side, thereby cooling the conductive element 224 and delivering cold therapy to the user's skin. The hot side of the thermoelectric cooler may be thermally coupled to a heat dissipation structure, such as a heat sink, cooling fins, or a thermally conductive housing portion of the lower cap assembly 200, to dissipate the heat generated during cooling operation. The circuit board 228 may include control circuitry configured to regulate the direction and magnitude of the electric current supplied to the thermoelectric cooler, thereby enabling the device to switch between a heating mode and a cooling mode. In the heating mode, the current direction may be reversed such that the side adjacent to the conductive element 224 becomes the hot side, delivering heat therapy to the user's skin. The button 232 may be configured to allow the user to select between heating and cooling modes, or the control circuitry may automatically alternate between modes based on a predetermined treatment protocol. This dual-mode capability allows the thermal element to provide both heat therapy and cold therapy using a single semiconductor component, enhancing the versatility of the functional cap.

In an embodiment, the stimulation element can be a phototherapy element that includes a light-emitting unit 226 (such as one or more LEDs, a light-emitting element, or a light source) and a light-transmitting region. The LEDs can emit at least one of red light, blue light, or infrared light. A portion of the lower cap assembly 200 can be formed as a light-transmitting region so that light emitted from the phototherapy element can exit through the light-transmitting area and be applied to the user's skin.

In an embodiment, the stimulation element can be a microcurrent element, which comprises a pair of electrodes and a microcurrent generator. The conductive element 224 can act as the microcurrent providing electrodes. When the electrodes contact the user's skin, the electrodes, the skin, and the microcurrent generator together may define a closed electrical loop. The microcurrent generator is configured to output and regulate a microcurrent within the loop, thereby delivering microcurrent stimulation to the user through the electrodes.

In an embodiment, the stimulation element can be an ultrasonic wave therapy element, which may comprise an ultrasonic transducer mounted on the circuit board 228. The ultrasonic transducer can be configured to emit ultrasonic waves through a contact surface of the lower cap assembly 200, such as through the first cover 208 or a dedicated ultrasonic transmission window. The ultrasonic waves may provide a massage effect to the user's skin, promote blood circulation, or enhance the absorption of skincare products by facilitating penetration of active ingredients into the skin. The ultrasonic transducer may operate at frequencies suitable for cosmetic applications, and the circuit board 228 may include drive circuitry configured to control the frequency and intensity of the ultrasonic output.

In an embodiment, the stimulation element can be a magnetotherapy element, which may comprise one or more permanent magnets or electromagnets disposed within the lower cap assembly 200. The magnets may be positioned within the mounting cavity 216 or integrated into the first cover 208 such that a magnetic field is generated at or near the contact surface of the lower cap assembly 200. When the magnetotherapy element is brought into proximity with the user's skin, the magnetic field may provide therapeutic benefits such as promoting circulation or relaxation. In configurations employing electromagnets, the electromagnets may be electrically connected to the circuit board 228, and the battery 230, and the button 232 may be configured to control activation and deactivation of the magnetotherapy function. The magnetotherapy element may be used independently or in combination with other stimulation elements such as heat therapy, phototherapy, or microcurrent therapy.

Referring now to FIG. 7, the lower cap assembly 200 includes a first cover 208 and a second cover 212 that cooperate to form a mounting cavity 216. A circuit board 228 and a battery 230 are accommodated within the mounting cavity 216. To enhance the positional stability of the battery 230, the mounting cavity 216 further defines a positioning groove 218 into which the battery 230 is fitted. The circuit board 228 is annular and extends around the clearance hole 206. The heating resistor 222 and the light-emitting unit 226 are mounted on the circuit board 228.

The first cover 208 has a pair of through holes 210. The conductive element 224 is fixed within each through hole 210. Each conductive element 224 is disposed directly opposite the corresponding heating resistor 222, thereby enabling efficient and rapid transfer of heat generated by the heating resistor 222 to the exterior surface of the device. The first cover 208 is made of a light-transmitting material, such that light emitted by the light-emitting unit 226 can pass through the first cover 208.

To conceal internal components and reduce visual exposure of the internal structure, the lower cap assembly 200 additionally includes a light shield 220 that covers the surface of the circuit board 228 facing the through holes 210. The light shield 220 is provided with openings corresponding to the positions of the heating resistor 222 and the light-emitting unit 226, allowing heat and light to be transmitted outward while maintaining the internal appearance of the device.

Furthermore, the second cover 212 is provided with a button 232 that is electrically connected to the circuit board 228. The button 232 is configured to control the activation and deactivation of the heat therapy element and the phototherapy element.

Referring to FIGS. 1 to 4, in an embodiment, the second end 204 (i.e., the lower end) of the lower cap assembly 200 is configured to cover the container mouth of the storage container 300, with the lower cap assembly 200 and the storage container 300 being secured together via mating internal and external threaded structures. In alternative embodiments, the lower cap assembly 200 may be directly fitted onto the neck of the storage container 300 and retained in place using snap-fit clips or equivalent fastening features.

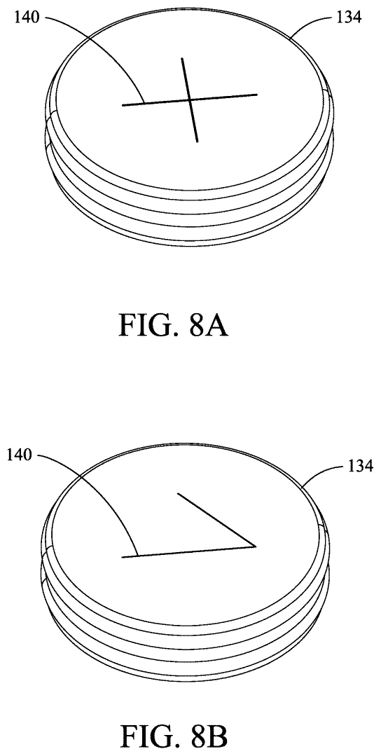

Referring to FIGS. 8A and 8B, in an embodiment, the leakage protection member comprises a retractable structure with one or more slits configured to allow controlled deformation. In this embodiment, the sealing ring 134 is provided with a closed bottom surface configured to seal the lower opening of the clearance hole 206 temporarily. The closed bottom surface prevents the skincare product from leaking through the clearance hole 206 when the upper cap assembly 100 is separated from the lower cap assembly 200 or when the cream applicator 138 is not engaged. To allow functional movement of the cream applicator 138 while maintaining sealing performance, the closed bottom surface of the sealing ring 134 is formed with a retractable slit/cut 140 structure. For example, the closed bottom surface may include a plus-sign-shaped slit/cut 140 formed by two intersecting slits, thereby creating four elastically deformable flaps. In other embodiments, the closed bottom surface may include a V-shaped slit/cut 140 comprising two slits defining a hinged flap capable of deflecting upward when the cream applicator 138 passes through. These slit configurations provide a retractable sealing surface that deforms to allow axial movement of the cream applicator 138 and returns to a closed state when the applicator is withdrawn, thereby maintaining both usability and leakage prevention.

The slits/cuts 140 are not limited to the plus-sign shape or the V-shape, the slit/cut 140 may be formed in any suitable shape, including curved, linear, multi-segment, star-shaped, or irregular geometries, so long as the resulting structure allows the closed bottom surface to elastically deform and retract when the cream applicator 138 passes through, and to return to a closed position when the applicator is withdrawn. This flexibility in cut design ensures that the sealing ring 134 can be adapted to various material properties, manufacturing methods, and sealing requirements without departing from the scope of the invention.

In use, the sealing ring 134 operates as a dynamic sealing component that maintains closure of the clearance hole 206 while still permitting passage of the cream applicator 138. When the cream applicator 138 is positioned above the sealing ring 134, the closed bottom surface of the sealing ring 134 remains in its natural, sealed state, with the slit/cut 140 defined flaps lying flat to block the opening and prevent the skincare product from leaking out. As the cream applicator 138 moves axially downward toward the clearance hole 206, its tip presses against the flaps formed by the cut pattern. The flaps elastically deform and retract downward, allowing the cream applicator 138 to pass through the bottom surface with minimal resistance. Once the cream applicator 138 passes through, the flaps flex around its outer surface, maintaining partial sealing engagement and reducing outward leakage.

When the cream applicator 138 is later withdrawn, the flaps return to their original position due to the elasticity and the elastic material of the sealing ring 134. The closed bottom surface is thereby re-established, sealing the hole once again. This automatic opening-and-closing behaviour enables the functional cap to maintain effective leakage prevention while still allowing smooth extension and retraction of the cream applicator 138 during operation.

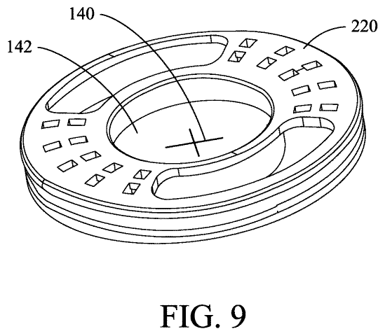

Referring to FIG. 9, in an alternate configuration, the lower cap assembly 200 can integrate a leakage protection member in the form of an in-built and additional sealing member 142, which can be disposed at the clearance hole 206. The sealing member 142 covers the opening of the clearance hole 206 and is anchored or attached along the corner regions defining the perimeter of the clearance hole 206. The sealing member 142 functions as a flexible barrier configured to prevent leakage of the skincare product through the clearance hole 206 when the cream applicator 138 is not present or when the upper cap assembly 100 and lower cap assembly 200 are separated. The sealing member 142 may be formed of an elastically deformable material that enables repeated bending, flexing, and restoration without structural failure.

Furthermore, the sealing member 142 is designed as a retractable structure incorporating one or more slits/cuts 140 patterns that allow controlled deformation. These slit/cut 140 patterns may include a plus-sign-shaped cut formed by intersecting slits, or a V-shaped cut formed by two angled slits, each defining flap regions capable of opening when the cream applicator 138 passes through. During insertion or withdrawal of the cream applicator 138, the flaps deform to permit movement and subsequently return to their closed state once the applicator is removed. In this manner, the sealing member 142 maintains an effective seal over the clearance hole 206, even when the cream applicator 138 is completely detached, ensuring reliable containment of the skincare product within the storage container 300.

In an embodiment, the upper cap assembly 100 is further configured to serve as an independent light-emitting element. The upper cap assembly 100 may incorporate one or more LEDs, a dedicated circuit board, and a rechargeable battery, enabling the upper cap assembly 100 to deliver phototherapy such as red light, blue light, or infrared light directly to the user's skin. The integration of these components allows the upper cap assembly 100 to function as a standalone skincare tool even when it is detached from the lower cap assembly 200, thereby enhancing the versatility and therapeutic capability of the device.

To facilitate recharging of the upper cap assembly 100, the bottom surface of the upper cap assembly 100 can be provided with a pair of charging electrodes. These electrodes are positioned such that, when the upper cap assembly 100 is coupled to the lower cap assembly 200, the electrodes correspond to and electrically interface with the conductive elements 224 of the lower cap assembly 200. In an embodiment, the conductive elements 224 are electrically conductive and are connected to the power circuitry housed within the lower cap assembly 200. As a result, the conductive elements 224 can function not only as thermal transfer elements but also as charging terminals capable of transferring electrical power to the charging electrodes of the upper cap assembly 100.

Furthermore, the dual-function nature of the conductive elements 224 allows them to deliver microcurrent stimulation when required. When operated in microcurrent mode, the conductive elements 224 act as output electrodes through which a controlled microcurrent is applied to the skin. When the upper cap assembly 100 is docked onto the lower cap assembly 200, these same conductive covers supply charging power to the upper cap's internal battery. This shared structural and electrical arrangement minimizes component redundancy, simplifies manufacturing, and enables seamless switching between charging and treatment functions.

In an embodiment, the applicator itself may be equipped with a light-therapy element configured to pre-treat the skincare product while it remains inside the container. This pre-treatment may enhance ingredient activation, improve formulation stability, or promote deeper absorption when subsequently applied to the skin.

In an embodiment, the lower cap assembly can further include an elastic locking member configured to selectively open and close the clearance hole 206. The elastic locking member performs the function of the leakage protection member to prevent the leakage of cosmetic from the storage container when the upper cap assembly is removed. The elastic locking member can be formed of an elastically deformable material such as silicone, thermoplastic elastomer, or thin polymer film, and is positioned at the clearance hole 206 to act as a dynamic sealing barrier. A pressing member can be operably coupled to the elastic locking member. When the pressing member is actuated by the user, the elastic locking member deforms, retracts, or deflects away from the clearance hole 206, thereby exposing the clearance hole 206 and permitting the cream applicator to pass through and access the skincare product within the container.

When the pressing member is released and returns to its resting position, the elastic locking member correspondingly returns to its natural, undeformed state due to its inherent elasticity. In this resting state, the elastic locking member covers and seals the clearance hole 206, preventing the skincare product from leaking out of the container even when the device is tilted or carried in a bag. This structure enables controlled access to the container contents only when manual pressure is applied to the pressing member, thereby improving leakage prevention, hygiene, and user control.

The pressing member can be located on the outer surface of the lower cap assembly 200 and may be configured as a button, tab, lever, or flexible press region. The coordinated action of the pressing member and the elastic locking member provides a simple, reliable mechanism for opening and re-sealing the clearance hole 206, enhancing both usability and product containment.

In an embodiment, the lower cap assembly 200 may include a skin condition sensor configured to detect one or more skin parameters such as moisture level, temperature, or impedance. The skin condition sensor may be mounted on the circuit board 228 and positioned to contact the user's skin through the first cover 208 or a dedicated sensor window. The sensor data may be used to adjust the intensity or duration of the stimulation elements, or to provide feedback to the user via an indicator light, audible signal, or a connected mobile application. In some configurations, the skin condition sensor may detect the skin condition before and after product application, allowing the user to assess the effectiveness of the skincare treatment.

In an embodiment, the functional cap may include a product-level sensor configured to detect the remaining level of skincare product in the storage container 300. The product level sensor may be an optical sensor, a capacitive sensor, or a weight-based sensor disposed within the lower cap assembly 200 or integrated into the storage container 300. The product level sensor may be electrically connected to the circuit board 228 and configured to provide a visual indication, such as an LED color change, when the product level falls below a predetermined threshold. This feature allows the user to anticipate when a refill or replacement is needed.

In an embodiment, the lower cap assembly 200 may include a wireless communication module configured to enable connectivity with an external device such as a smartphone or tablet. The wireless communication module may employ Bluetooth, Bluetooth Low Energy (BLE), Near Field Communication (NFC), Wi-Fi, or other suitable wireless protocols. The wireless communication module may be electrically connected to the circuit board 228 and configured to transmit data such as usage history, treatment duration, selected modes, or skin sensor readings to a mobile application. The mobile application may provide features such as usage tracking, personalized skincare recommendations, treatment protocol customization, or reminder notifications.

In an embodiment, the functional cap may include a smart reminder system configured to prompt the user for regular skincare routines. The smart reminder system may include a timer circuit on the circuit board 228 and one or more feedback elements such as LEDs, a vibration motor, or an audible buzzer. The smart reminder system may be configured to activate the feedback elements at predetermined intervals or at user-specified times to remind the user to apply skincare products or perform stimulation treatments. The reminder intervals and feedback preferences may be adjustable via the button 232 or through a connected mobile application.

In an embodiment, the cream applicator 138 may be detachably connected to the slider 126, allowing the user to interchange different applicator heads depending on the type of skincare product or application preference. The detachable connection may be achieved through a snap-fit engagement, threaded coupling, magnetic attachment, or bayonet mount. Interchangeable applicator heads may include scoops of different sizes, brushes with varying bristle densities, rollers, sponge tips, or silicone pads. This modular applicator configuration allows the user to customize the application experience and facilitates cleaning or replacement of worn applicator heads.

In an embodiment, the cream applicator 138 may include configured to enhance product distribution and provide additional massage effect during application. A small motor may be disposed within the slider 126 or the upper cap assembly 100 and operably connected to the cream applicator 138. The motor may be electrically connected to the circuit board 228 or a separate power source within the upper cap assembly 100. The rotation or vibration may be activated by a dedicated button or automatically upon detection of contact with the skin or skincare product.

In an embodiment, the sealing structure at the clearance hole 206 may comprise an iris diaphragm mechanism instead of or in addition to the sealing ring 134 having a slit. The iris diaphragm may include a plurality of overlapping blades arranged in a circular pattern, the blades being configured to open and close in response to axial movement of the cream applicator 138 or actuation of a control ring. The iris diaphragm provides an adjustable aperture size and enhanced sealing performance, and may be formed of flexible polymer material or thin metal blades.

In an embodiment, the sealing member 142 or sealing ring 134 may include embedded magnetic elements configured to provide automatic closure when the cream applicator 138 is withdrawn. The magnetic elements may be permanent magnets or magnetized portions of the sealing material arranged such that magnetic attraction draws the sealing surfaces together when the cream applicator 138 is not present. This magnetic sealing configuration provides reliable closure without relying solely on material elasticity.

In an embodiment, the upper cap assembly 100 or lower cap assembly 200 may include a solar charging panel integrated into an outer surface thereof. The solar charging panel may be electrically connected to the battery 230 and configured to provide supplemental charging when exposed to ambient light or sunlight. The solar charging panel may be a thin-film photovoltaic cell or a flexible solar panel conforming to the curved surface of the cap assembly.

In an embodiment, the lower cap assembly 200 may include an inductive charging coil configured to enable wireless charging on a standard wireless charging pad. The inductive charging coil may be disposed within the mounting cavity 216 and electrically connected to the battery 230 through charging circuitry on the circuit board 228. This configuration allows the user to charge the device by simply placing it on a compatible wireless charging surface without requiring physical connection of cables or alignment of charging electrodes.

In an embodiment, the lower cap assembly 200 may include a USB-C charging port or other wired charging interface disposed on an outer surface of the second cover 212. The USB-C port may be electrically connected to the battery 230 through charging circuitry on the circuit board 228 and may be protected by a removable cover or sealed gasket to prevent ingress of moisture or skincare product.

In an embodiment, the stimulation element may include a vibration massage element comprising a vibration motor disposed within the lower cap assembly 200. The vibration motor may be mounted on the circuit board 228 or within the mounting cavity 216 and configured to generate vibrations that are transmitted through the first cover 208 or conductive element 224 to the user's skin. The vibration massage element may operate independently or in combination with other stimulation elements, such as heat therapy or phototherapy, to enhance skincare absorption and provide a relaxing massage effect.

In an embodiment, the stimulation element may include an ionic therapy element configured to generate positive or negative ions for enhanced skincare product penetration. The ionic therapy element may comprise one or more ion-emitting electrodes disposed on the first cover 208 or integrated into the conductive element 224. The ion-emitting electrodes may be electrically connected to the circuit board 228, which includes circuitry configured to generate the ionic output. The polarity of the ionic output may be selectable by the user via the button 232 to match the charge characteristics of the skincare product being applied.

In an embodiment, the stimulation element may include a radio frequency (RF) element configured to emit radio frequency energy for deeper tissue stimulation and collagen promotion. The RF element may comprise one or more RF electrodes disposed on the first cover 208 and electrically connected to the RF generation circuitry on the circuit board 228. The RF element may operate at frequencies suitable for cosmetic applications and may include temperature monitoring to ensure safe operation.

In an embodiment, the stimulation element may include an electrical muscle stimulation (EMS) element configured to deliver electrical impulses to facial muscles for toning and lifting effects. The EMS element may comprise a pair of EMS electrodes disposed on the first cover 208 or integrated into the conductive elements 224. The EMS electrodes may be electrically connected to the EMS generation circuitry on the circuit board 228, which is configured to output controlled electrical impulses at frequencies and intensities suitable for facial muscle stimulation.

In an embodiment, the storage container 300 may comprise a dual-chamber configuration having two separate compartments for storing different skincare products. The dual-chamber container may include a dividing wall separating a first compartment from a second compartment, each compartment having its own opening accessible through the clearance hole 206. The cream applicator 138 may be configured to access either compartment, or two separate applicators may be provided. This configuration allows the user to store complementary products such as day cream and night cream, or serum and moisturizer, in a single container assembly.

In an embodiment, the storage container 300 may be configured as a refillable cartridge that is detachably connected to the lower cap assembly 200. The refillable cartridge may snap into, thread onto, or magnetically attach to the lower cap assembly 200, allowing the user to replace the cartridge when the skincare product is depleted without discarding the functional cap assembly. This configuration reduces waste and allows the user to easily switch between different skincare products by swapping cartridges.

In an embodiment, the lower cap assembly 200 may include a plurality of LED status indicators configured to display information such as battery 230 level, selected treatment mode, treatment progress, or device status. The LED status indicators may be disposed on an outer surface of the second cover 212 or visible through a translucent portion of the lower cap assembly 200. The LED status indicators may display different colors or blinking patterns to convey different information to the user.

In an embodiment, the lower cap assembly 200 may include a small display screen, such as an OLED display or LCD display, configured to show treatment information. The display screen may be disposed on an outer surface of the second cover 212 and electrically connected to the circuit board 228. The display screen may show information such as treatment time remaining, current temperature, selected mode, battery level, or usage statistics. The display screen may be controlled via the button 232 or through a connected mobile application.

In an embodiment, the circuit board 228 may include an auto-shutoff timer configured to automatically deactivate the stimulation elements after a predetermined treatment duration. The auto-shutoff timer may be set to a fixed duration or may be adjustable by the user via the button 232 or a connected mobile application. This safety feature prevents overuse of the stimulation elements and conserves battery power.

In an embodiment, the lower cap assembly 200 may include a temperature safety cutoff configured to monitor the temperature of the conductive element 224 or other thermal elements and automatically cut power if the temperature exceeds a predetermined safe threshold. The temperature safety cutoff may comprise a temperature sensor electrically connected to the circuit board 228, which includes control circuitry configured to interrupt power to the heating resistor 222 or thermoelectric cooler when an unsafe temperature is detected. This safety feature protects the user from burns or discomfort due to excessive heat.

In an embodiment, the outer surface of the lower cap assembly 200 may include a contoured grip surface configured to improve handling during stimulation treatment. The contoured grip surface may include textured regions, finger indentations, or ergonomic curves that conform to the user's hand. The contoured grip surface may be formed integrally with the second cover 212 or may be provided as a separate overmolded grip element. This ergonomic feature enhances user comfort and control during handheld operation of the stimulation elements.

In an embodiment, the upper cap assembly 100 and lower cap assembly 200 may be configured to nest or collapse into a more compact form for travel or storage. The compact travel configuration may involve telescoping components, foldable elements, or a nesting arrangement wherein the upper cap assembly 100 fits within a recess of the lower cap assembly 200 when not in use. This configuration reduces the overall size of the device for convenient portability in travel bags or cosmetic pouches.

In an embodiment, a method of applying a skincare product from a storage container using the functional cap is provided. The method comprises removing the upper cap assembly 100 from the lower cap assembly 200 of the functional cap, wherein the lower cap assembly 200 is mounted on the storage container 300 and includes at least one stimulation element. When the upper cap assembly 100 is removed, the cream applicator 138 extends from the upper cap assembly 100 by the elastic reset member 132 as the cream applicator 138 disengages from the bottom of the storage container 300. The elastic reset member 132 releases its stored elastic potential energy and urges the cream applicator 138 toward an extended position, thereby increasing the exposed length of the cream applicator 138.

The method further comprises scooping the skincare product from the storage container 300 using the extended cream applicator 138. The extended length of the cream applicator 138 allows the user to reach the bottom and sidewalls of the storage container 300, thereby minimizing residual product and enhancing overall utilization of the skincare formulation. The user may scoop a desired amount of skincare product onto the cream applicator 138 and withdraw the cream applicator 138 from the storage container 300. Notably, retrieving the skincare product using the cream applicator 138 occurs without direct finger contact with the skincare product, thereby improving hygiene and reducing the risk of contamination.

The method further comprises applying the skincare product to the skin. The user may transfer the skincare product from the cream applicator 138 to the desired area of the skin, such as the face, neck, or hands. The cream applicator 138 may be used to spread or distribute the skincare product evenly across the skin surface.

The method further comprises stimulating the skin using at least one stimulation element of the lower cap assembly 200 to promote absorption of the skincare product. The user may detach the lower cap assembly 200 from the storage container 300 and apply the stimulation element directly to the skin where the skincare product has been applied. The stimulation element may be activated by pressing the button 232 on the lower cap assembly 200.

In an embodiment, stimulating the skin comprises applying at least one of heat therapy, cold therapy, phototherapy, piezoelectric therapy, ionic therapy, vibration therapy, ultrasonic wave therapy, magneto-therapy or microcurrent therapy using at least one stimulation element. For heat therapy, the heating resistor 222 generates thermal energy that is transferred through the conductive element 224 to the user's skin, promoting blood circulation and enhancing absorption of the skincare product. For cold therapy, the thermoelectric cooler operates in cooling mode to absorb heat from the conductive element 224, delivering a cooling sensation to the skin that may reduce puffiness or soothe irritation. For phototherapy, the light-emitting unit 226 emits therapeutic light, such as red light, blue light, or infrared light, through the light-transmitting first cover 208 to the skin. For microcurrent therapy, the conductive elements 224 function as electrodes to deliver a controlled microcurrent to the skin, stimulating facial muscles and promoting cellular activity.

In an embodiment, stimulating the skin comprises transferring heat generated by the heating resistor 222 through the conductive element 224 to the skin. When the button 232 is pressed to activate the heat therapy element, electrical current flows through the heating resistor 222, causing it to generate thermal energy. The thermal energy is rapidly conducted through the conductive element 224, which is positioned in direct contact with or in proximity to the user's skin. The heat transferred to the skin promotes vasodilation, increases local blood flow, and enhances the penetration and absorption of the applied skincare product into the deeper layers of the skin.

The method may be performed in a sequential manner, wherein the user first applies the skincare product using the cream applicator 138 and then immediately stimulates the skin using the stimulation element of the lower cap assembly 200. This sequential application allows the stimulation treatment to be performed while the skincare product is still fresh on the skin, thereby maximizing the therapeutic benefits and absorption efficiency. Alternatively, the user may perform the stimulation treatment at a later time or may repeat the stimulation treatment multiple times to achieve the desired results. After completing the skincare application and stimulation treatment, the method may further comprise resealing the storage container 300 using the functional cap. The user may reattach the upper cap assembly 100 to the lower cap assembly 200 and remount the functional cap onto the storage container 300 to prevent drying, contamination, or spillage of the remaining skincare product.

The present invention provides a functional cap and storage container system that significantly enhances product extraction, hygiene, and therapeutic performance. Through its elastic reset cream applicator, detachable upper and lower cap assemblies, integrated stimulation elements, advanced sealing structures, and optional thermal therapy, light-therapy, microcurrent therapy, etc., the invention enables users to efficiently access, apply, and treat skincare products with improved convenience and reduced waste. The combination of retractable sealing components, guided sliding mechanisms, quick-release coupling, and optional charging interfaces results in a compact, versatile, and user-friendly design that resolves long-standing limitations of traditional skincare containers. This invention thereby offers a superior, value-added solution tailored to modern cosmetic and therapeutic needs.

The present invention exhibits broad industrial applicability across the personal care, cosmetic, dermatological, and home-use skincare device sectors. The functional cap can be implemented in mass-manufactured skincare products, premium cosmetic packaging, therapeutic applicators, and portable beauty devices. Its integrated applicator mechanism improves product utilization and hygiene, while the detachable lower cap assembly with embedded stimulation elements adds value by enabling thermal therapy, phototherapy, microcurrent therapy, or other skin-treatment modalities. The device is compatible with standard container-neck formats, can be produced using conventional injection-molding and electronic-assembly processes, and supports scalable manufacturing with low tooling complexity. As a result, the invention is well-suited for widespread commercial deployment in consumer skincare products, professional treatment devices, travel-friendly beauty kits, and multifunctional cosmetic packaging solutions.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims

1. A functional cap for a storage container, comprising:
a lower cap assembly defining a clearance hole extending therethrough and housing at least one stimulation element;
an upper cap assembly detachably connected to the lower cap assembly, the upper cap assembly positioned to cover the clearance hole when attached to the lower cap assembly;
a leakage protection member to form a fluid-resistant seal between a wall of the clearance hole and the upper cap assembly; and
a cream applicator extending through the clearance hole and supported by the upper cap assembly,
wherein the cream applicator is positioned to contact a skincare product stored in the storage container when the functional cap is mounted on the storage container, and wherein the lower cap assembly is configured to provide one or more therapies to a user through at least one stimulation element.

2. The functional cap of claim 1, wherein the lower cap assembly has opposite first and second ends, the clearance hole extending between the first end and the second end.

3. The functional cap of claim 2, wherein at least one stimulation element is disposed at the first end of the lower cap assembly, and the upper cap assembly covers the first end.

4. The functional cap of claim 1, wherein the leakage protection member is a sealing ring disposed at the clearance hole, the sealing ring comprises an annular elastomeric body configured to inhibit leakage of the skincare product.

5. The functional cap of claim 1, wherein the cream applicator includes an applicator surface selected from a spatula, pad, brush, or scoop.

6. The functional cap of claim 1, wherein the leakage protection member comprises a retractable structure with one or more slits configured to allow controlled deformation.

7. The functional cap of claim 1, wherein the leakage protection member comprises an elastic locking member disposed at the clearance hole and configured to selectively open and close the clearance hole; and a pressing member operably coupled to the elastic locking member, wherein actuation of the pressing member causes the elastic locking member to deform and expose the clearance hole, and release of the pressing member causes the elastic locking member to return to a closed state covering the clearance hole.

8. The functional cap of claim 1, wherein:
the upper cap assembly includes a rechargeable battery and a pair of charging electrodes disposed on a bottom surface of the upper cap assembly; and
the lower cap assembly includes a pair of conductive elements electrically connected to power circuitry housed within the lower cap assembly,
wherein the pair of charging electrodes are positioned to electrically interface with the pair of conductive element when the upper cap assembly is coupled to the lower cap assembly, such that the pair of conductive element act as charging terminals to transfer electrical power to the rechargeable battery of the upper cap assembly.

9. The functional cap of claim 1, further comprising an elastic reset member disposed within the upper cap assembly and operably connected to the cream applicator.

10. The functional cap of claim 9, wherein the elastic reset member comprises a spring or elastomeric element, the elastic reset member permits axial displacement of the cream applicator relative to the upper cap assembly.

11. The functional cap of claim 9, wherein the upper cap assembly comprises an upper cover body, a sleeve, and a slider, the upper cover body having a slot, one end of the sleeve being inserted into the slot, the elastic reset member and the slider being located within the sleeve, the slider being slidably engaged with the sleeve, and one end of the cream applicator being fixed to the slider.

12. The functional cap of claim 11, wherein an inner wall of the sleeve is provided with a plurality of guide ribs extending along an axial direction of the sleeve, and a circumferential surface of the slider is provided with a plurality of guide grooves configured to receive the plurality of guide ribs in a one-to-one correspondence.

13. A skincare product dispensing and treatment system, comprising:
a storage container configured to hold a skincare product; and
a functional cap removably coupled to the storage container;
wherein the functional cap includes a lower cap assembly housing at least one stimulation element, an upper cap assembly detachably connected to the lower cap assembly, a leakage protection member disposed at a clearance hole of the lower cap assembly, and a cream applicator supported by the upper cap assembly and extending through the clearance hole into the storage container;
wherein the lower cap assembly is separable from the storage container and configured to provide one or more therapies to a user through at least one stimulation element.

14. The storage container of claim 13, the leakage protection member is a sealing ring configured to seal a gap between a wall of the clearance hole and the upper cap assembly.

15. The storage container of claim 13, wherein the lower cap assembly further comprises a first cover and a second cover that cooperate to form a mounting cavity, the mounting cavity is configured to accommodate a circuit board and a battery electrically connected to at least one stimulation element.

16. The storage container of claim 15, wherein the at least one stimulation element comprises a heating resistor mounted on the circuit board and a conductive element fixed in a through hole of the first cover, the conductive element being positioned directly opposite the heating resistor to transfer heat generated by the heating resistor to a user's skin.

17. The storage container of claim 15, wherein the first cover is formed of a light-transmitting material, and at least one stimulation element comprises a light-emitting element mounted on the circuit board and configured to emit therapeutic light through the first cover.

18. A method of applying a skincare product and providing skin treatment using a functional cap and a storage container, the method comprising:
providing an upper cap assembly in the functional cap, the upper cap assembly comprising a cream applicator to contact a skincare product stored in the storage container;
providing a lower cap assembly in the functional cap, the lower cap assembly having a clearance hole for allowing the cream applicator to pass through, the lower cap assembly housing at least one stimulation element;
providing a leakage protection member configured to close the clearance hole to prevent leakage of the skincare product;
removing the functional cap from the storage container;
retrieving the skincare product from the storage container using the cream applicator;
applying the skincare product to the skin using the cream applicator; and
applying one or more therapies to a user using at least one stimulation element of the lower cap assembly.

19. The method of claim 18, wherein applying one or more therapies is performed after application of the skincare product.

20. The method of claim 18, wherein the cream applicator is biased toward an extended position by an elastic reset member.
